(19) United States
(12) Patent Application Publication
(10) Pub. No.: US 2019/0212232 A1
(43) Pub. Date: Jul. 11, 2019

(54) PORTABLE BIO-AEROSOL COLLECTION DEVICE AND ANALYSIS METHOD

(71) Applicants: University-Industry Foundation, Yonsei University, Seoul (KR); Research Cooperation Foundation of Yeungnam University, Gyeongsangbuk-do (KR)

(72) Inventors: Jung Ho Hwang, Seoul (KR); Ji Woon Park, Seoul (KR); Hyeong Rae Kim, Seoul (KR); Jeong Hoon Byeon, Daegu (KR)

(21) Appl. No.: 16/328,766

(22) PCT Filed: Sep. 12, 2017
(86) PCT No.: PCT/KR2017/010015

§ 371 Date: Feb. 27, 2019

(30) Foreign Application Priority Data
Sep. 12, 2016 (KR) 10-2016-0116949

(51) Int. Cl. G01N 1/22; G01N 1/14; C12Q 1/6806; C12Q 1/6872; C12Q 1/06

(52) U.S. Cl. CPC G01N 1/22; G01N 1/14; C12Q 1/06; C12Q 1/6872; C12Q 1/6806

(57) ABSTRACT

The present invention relates to a portable bioaerosol collecting device and an analysis method using the same, and particularly, to a portable bioaerosol collecting device, which includes a first body comprising an air inlet, a second body coupled to the first body and including an air outlet, and a collecting region created by coupling the first and second bodies together.

### CROSS-REFERENCE TO RELATED APPLICATION

This application claims priority to and the benefit of Korean Patent Application No. 10-2016-0116949, filed on Sep. 12, 2016, the disclosure of which is incorporated herein by reference in its entirety.

### BACKGROUND

### 1. Field of the Invention

The present invention relates to a portable bioaerosol collecting device and a method of analyzing bioaerosols using the same.

### 2. Discussion of Related Art

A conventional device (impactor) for collecting bioaerosols (bacteria, fungi, etc.) needs inconvenient tasks of preparing agar plates for collection and culturing the collected microorganisms (for a maximum of two weeks or more). In addition, the conventional device (impactor) for detecting microorganisms in air using a collision method has various design variables, is difficult be processed and is expensive.

### SUMMARY OF THE INVENTION

The present invention is directed to providing a collecting device which is small and simply processed, and can be easily and simply used by common people without special expertise and applied to various analysis devices by easily and effectively collecting all types of microorganisms in air, and an analysis method using the same.

One aspect of the present invention provides a portable bioaerosol collecting device, which includes: a first body including an air inlet; a second body coupled to the first body and including an air outlet; and a collecting region created by coupling the first and second bodies.

In the device according to the present invention, the first or second body may further include a fixing means for fixing the coupling of the first and second bodies.

In the present invention, the fixing means may include a latch engaging with a latching jaw formed at the first or second body by latch-coupling, and a lever for forming and releasing the latch-coupling of the latch.

In the present invention, the fixing means may include screw holes formed in the first and second bodies, and screws for screw-coupling with the screw holes.

In the present invention, the collecting region may be created by coupling a concave part formed in the coupling surface between the first and second bodies.

The device according to the present invention may further include a collecting tool inserted into the collecting region.

In the present invention, the collecting tool may be a cotton swab or a filter.

In the present invention, the first body may include an air hole connecting the air inlet and the collecting region, and the second body may include an air hole connecting the collecting region and the air outlet.

In the present invention, the first and second bodies may include grooves formed in the respective coupling surfaces, and the device according to the present invention may further include a sealing means inserted into each groove.

In the present invention, the sealing means may be a rubber ring.

The device according to the present invention may further include a pump connected to the air outlet or the air inlet.

The device according to the present invention may have a collection efficiency (E_{collection}), represented by Mathematical Formula 1, in a range of 95 to 100%.

E_{collection}=[1−(N_{after}/N_{before})]×100 [Mathematical Formula 1]

In Mathematical Formula 1, N_{before} refers to a microbial concentration (#/m³) in the front section of the collecting device, and N_{after} refers to a microbial concentration (#/m³) in the rear section of the collecting device.

The device according to the present invention may maintain the collection efficiency (95 to 100%) even at a high flow rate of 100 to 200 L/min.

In addition, the present invention provides a method of analyzing bioaerosols, which includes: collecting bioaerosols by inputting air onto a bioaerosol collecting tool which is placed in a collecting region in the portable bioaerosol collecting device described above; and analyzing the bioaerosols collected on the bioaerosol collecting tool.

In the present invention, the inputting of air may be performed using the pump.

In the analysis step according to the present invention, only bioaerosols may be extracted from the collecting tool using a lysis reagent, and then light intensity may be measured, thereby quantifying microorganisms.

In the present invention, light intensity may be measured using an adenosine triphosphate (ATP) bioluminescence kit.

In the analysis step according to the present invention, the collecting tool may be washed with a liquid, and then the liquid may be subjected to mass spectrometry.

In the present invention, the mass spectrometry may be performed using a Matrix-Assisted Laser Desorption and Ionization-Time of Flight (MALDI-TOF) mass spectrometer.

In the analysis step according to the present invention, the collecting tool may be washed with a liquid, and then the liquid may be subjected to polymerase chain reaction (PCR) analysis.

In the analysis step according to the present invention, the collecting tool may be washed with a liquid, and then the liquid may be plated on an agar plate and subjected to quantitative analysis.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows each of a plan view of a first body of a collecting device according to a first exemplary embodiment of the present invention and a cross-sectional view of the device;

FIG. 2 shows each of a cross-sectional view of a first body and a plan view of a second body of the collecting device according to a first exemplary embodiment of the present invention;

FIG. 3 is a perspective view of a collecting device according to a second exemplary embodiment of the present invention;

FIGS. 4 and 5 are schematic diagrams illustrating collecting and analysis of microorganisms according to the present invention, respectively; and

FIG. 6 shows SEM images taken before and after bioaerosols are collected on a collecting tool in a collecting device according to an exemplary embodiment of the present invention.

### DETAILED DESCRIPTION OF EXEMPLARY EMBODIMENTS

Hereinafter, the present invention will be described with reference to the accompanying drawings.

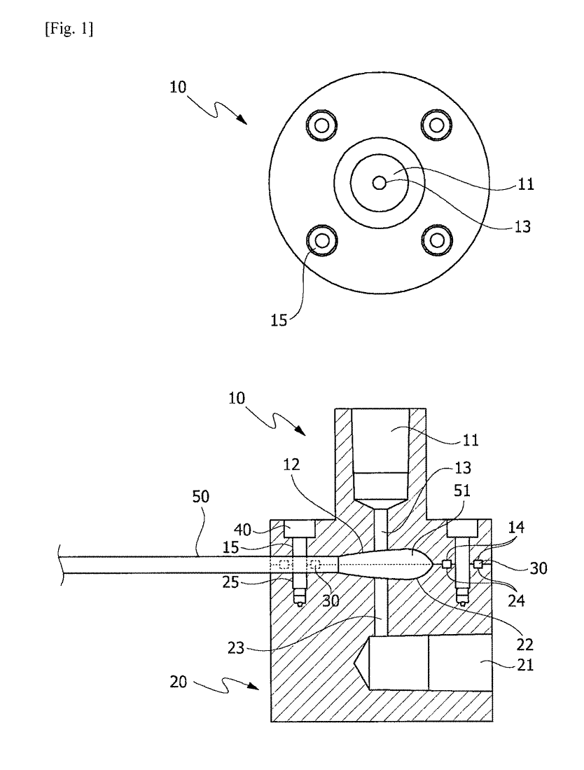

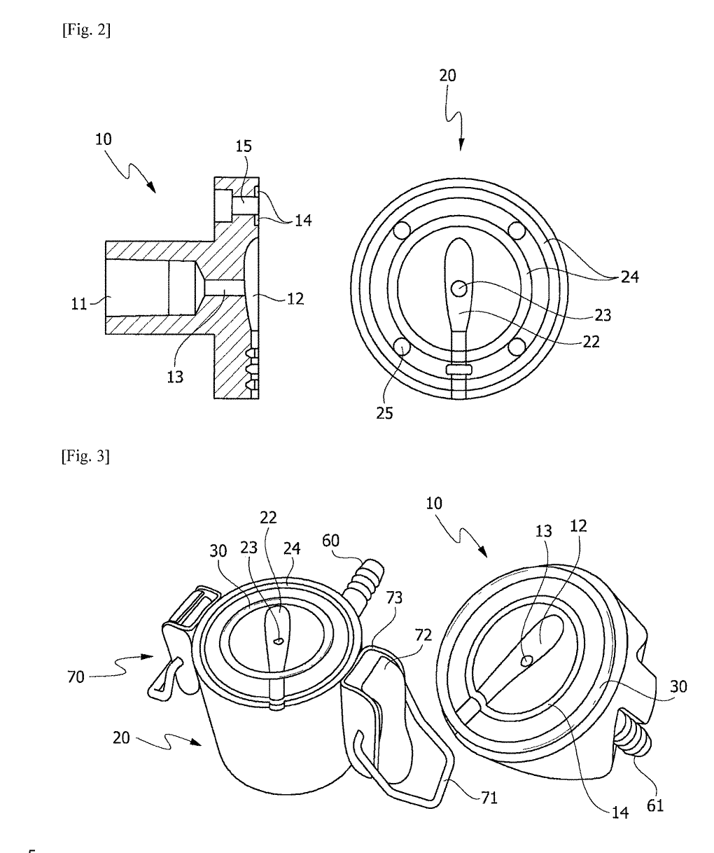

FIG. 1 shows each of a plan view of a first body of a collecting device according to a first exemplary embodiment of the present invention and a cross-sectional view of the device, and FIG. 2 shows each of a cross-sectional view of a first body and a plan view of a second body of the collecting device according to a first exemplary embodiment of the present invention.

The collecting device according to an exemplary embodiment may be largely divided into a first body 10, a second body 20, a sealing means 30, a fixing means 40, and a collecting tool 50.

The first body 10 corresponds to a lid disposed at an upper side, and may be manufactured of a metal or plastic, and formed in a cylindrical shape.

An air inlet 11 may be formed in an upper side or a side surface of the first body 10, and air may be introduced from the air inlet 11. As shown in FIG. 3, a connecting tube 61 may be inserted into the air inlet 11.

A concave part 12 may be formed in the bottom surface, which is a coupling surface of the first body 10. The concave part 12 of the first body 10 may be coupled to a concave part 22 of the second body 20 to form a collecting region and the collecting tool 50 may be inserted into the collecting region. The concave part 12 may be formed so as to have an upper shape and size of the collecting tool 50 to engage with the upper part of the collecting tool 50. In the drawing, a cotton swap is illustrated as the collecting tool 50, and the concave part 12 is also formed in the form of a cotton swab.

In the first body 10, an air hole 13 connecting the air inlet 11 with the concave part 12, which is a collecting region, may be formed. The air introduced from the air inlet 11 may flow to the concave part 12 through the air hole 13.

One or multiple grooves 14 may be formed in the bottom surface, which is a coupling surface, of the first body 10. A sealing means 30 such as a rubber ring may be inserted into the groove(s) 14. The groove(s) 14 may be formed in a circular shape, and formed so as to have the upper shape and size of the sealing means 30 to engage with the sealing means 30.

A screw hole 15 coupled with a screw 40, which is a fixing means, may be formed in the first body 10. One or preferably multiple screw holes 15 are formed. The screw hole 15 may be threaded. For screw-coupling, the upper side of the first body 10 may be recessed, or the lower side of the first body may protrude. In other words, the upper part of the first body 10 has a smaller diameter than the lower part thereof.

The second body 20 may be disposed at a lower part of the device, correspond to a support on which the collecting tool 50 is placed, and may be formed of a metal or plastic to have a cylindrical shape.

An air outlet 21 may be formed in a side or lower part of the second body 20, and air may be discharged through the air outlet 21. As shown in FIG. 3, a connecting tube 60 may be inserted into the air outlet 21.

The concave part 22 may be formed in the top surface, which is a coupling surface of the second body 20. As described above, the concave part 22 of the second body 20 may be coupled to the concave part 12 of the first body 10, thereby forming a collecting region in which the collecting tool 50 is inserted. The concave part 22 may be formed so as to have the lower shape and size of the collecting tool 50 to engage with the lower part of the collecting tool 50.

An air hole 23 for connecting the air outlet 21 with the concave part 22, which is a collecting region, may be formed in the second body 20. The air introduced from the concave part 22 may flow through the air hole 23 to the air outlet 21.

One or multiple grooves 24 into which a sealing means 30 such as a rubber ring is inserted may be formed in the top surface, which is the coupling surface, of the second body 20. The groove(s) 24 may be formed in a circular shape, and formed so as to have the lower shape and size of the sealing means 30 to engage with the sealing means 30.

One or multiple screw holes 25 coupled to the screw 40, which is a fixing means, may be formed in the second body 20.

The sealing means 30 may be inserted into the grooves 14 and 24 of the first body 10 and the second body 20 to improve sealing performance of the collecting device. As the sealing means 30, a rubber ring may be used. As shown in the drawing, multiple sealing means 30 may be disposed at the outermost part and the inside part of the device to at least doubly or triply seal the device, thereby improving sealing performance.

The screw 40, as a fixing means, may be coupled to the screw holes 15 and 25 of the bodies 10 and 20, thereby fixing each of the bodies 10 and 20.

The collecting tool 50 may be inserted into the collecting region to collect bioaerosols.

The bioaerosols may be defined as a state in which biological factors are dispersed as fine particles in a gaseous environment, and may include debris or toxins and particulate matter which are discharged from microorganisms and various living organisms. As the collecting tool 50, a cotton swab or a filter may be used, and preferably a cotton swab may be used. The cotton swab may be easily obtained in the surroundings, simply installed in the collecting device, and may have excellent collection efficiency. The cotton swab may consist of a supporting rod formed of wood or plastic, and a filter part attached to an end of the rod. Like the cotton swab, a collecting section 51 consisting of a filter part may be included at the end of the collecting tool 50.

In addition, the collecting device according to the present invention may include a pump (not shown). The pump is the source of the air flow, which allows the inflow of air into the collecting device and then discharges the air. The pump may be connected with the air outlet 21, the air inlet 11 or connecting tubes 60 and 61 by a tube or pipe.

FIG. 3 is a perspective view of a collecting device according to a second exemplary embodiment of the present invention, which has a fixing means different from that of the first exemplary embodiment of the present invention. That is, each body 10 or 20 may be fixed by latch-coupling, rather than screw-coupling. Other components except the fixing means may be equivalent or similar to those of the first exemplary embodiment of the present invention, and thus detail descriptions of these components will be omitted.

A fixing means 70 of the second exemplary embodiment may include a latch 71, a lever 72 and a fixing bracket 73, which are similar to those used in a lunch box or food container. The fixing means 70 may be installed on the second body 20 as shown in the drawing, or alternatively installed on the first body 10. The latch 71 may be formed in a partially inclined ring shape, and may engage with a latching jaw (not shown) formed on the first body 10 or the second body 20 by latch-coupling. The lever 72 may be connected to the latch 71, thereby forming and releasing the latch-coupling of the latch 71. The fixing bracket 73 is fixed to the second body 20 or the first body 10, and the latch 71 and the lever 72 may be pivotally mounted on the fixing bracket 73. When the latch 71 is raised to near the latching jaw, the lever 72 is also raised, and then, when the lever 72 is pulled downward, the latch 71 and the latching jaw are in close contact with each other, thereby forming the latch-coupling. On the contrary, when the lever 72 is raised, the latch-coupling may be released.

The collecting device according to the present invention may have excellent collection efficiency, and specifically, the collection efficiency (E_{collection}), represented by the following Mathematical Formula 1, may be in a range of 95 to 100%.

E_{collection}=[1−(N_{after}/N_{before})]×100 [Mathematical Formula 1]

In Mathematical Formula 1, N_{before} refers to a microbial concentration (#/m³) at the front section portion of the collecting device, and N_{after} refers to a microbial concentration (#/m³) at the rear section of the collecting device.

Particularly, the collecting device according to the present invention has excellent collection efficiency regardless of a flow rate, and may maintain a high collection efficiency (95 to 100%) at a high flow rate of, specifically, 100 to 200 L/min.

In addition, the present invention provides a method of analyzing bioaerosols. The analysis method according to the present invention may include collecting bioaerosols by inputting air onto a bioaerosol collecting tool which is placed in a collecting region in the portable bioaerosol collecting device described above; and analyzing the bioaerosols collected on the bioaerosol collecting tool.

First, in the collecting step, when a collecting tool such as a cotton swab serving as a filter is inserted into a collecting region of the microbial collecting device, and a pump is operated after being connected to an air outlet or a connecting tube, air is introduced through the air inlet and then flows to the collecting region through the air hole, and as the air collides with the collecting tool in the collecting region, the bioaerosols in the air are attached to the collecting tool and collected. Afterward, the rest of the air is discharged through the air outlet.

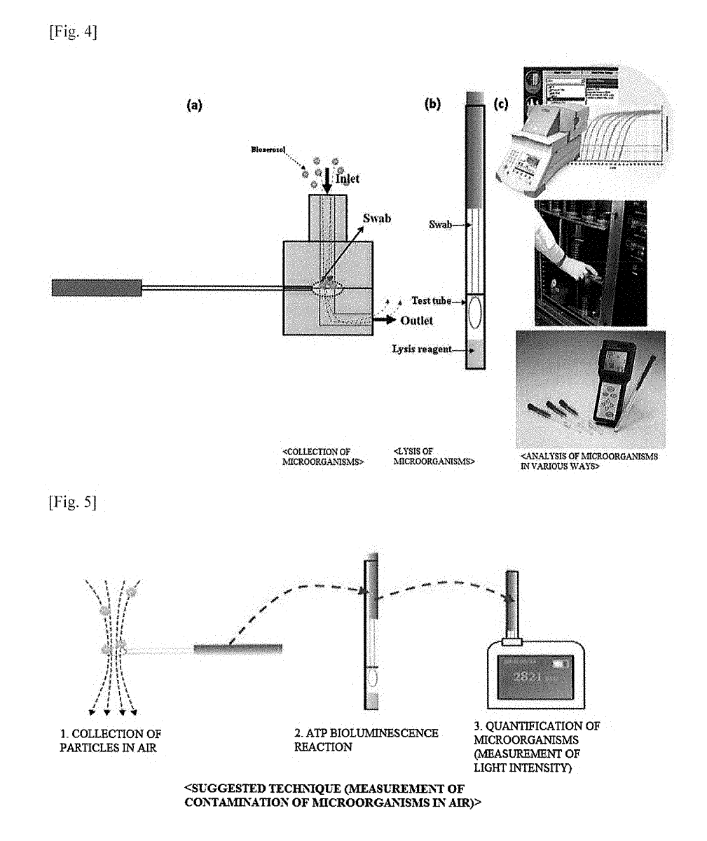

Then, as shown in FIGS. 4 and 5, the bioaerosols collected on the collecting tool may be analyzed in various ways, and for example, three analysis methods may be performed as below.

First, only bioaerosols may be extracted from the collecting tool using a lysis reagent, and then using liquid phase sample obtained from above procedure, light intensity is measured, thereby quantifying microorganisms. Only biocomponents such as microorganisms attached to the cotton swab are separated from the cotton swab by the lysis reagent, and dust remains on the collecting tool. The light intensity may be measured using a commercially available adenosine triphosphate (ATP) bioluminescence kit.

Second, the collecting tool is washed with a liquid (deionized water, etc.), and then the liquid obtained from the washing step may be subjected to mass spectrometry. The mass spectrometry may be performed using a MALDI-TOF (Matrix-Assisted Laser Desorption and Ionization-Time of Flight) mass spectrometer.

Third, the collecting tool is washed with a liquid (deionized water, etc.), and then the liquid obtained from the washing step may be subjected to polymerase chain reaction (PCR) analysis.

In addition, in the analysis step according to the present invention, after the collecting tool is washed with a liquid, the liquid obtained from the washing step may be plated on, for example, an agar plate and subjected to quantitative analysis, and a method of quantitative analysis after plating on the agar plate is known in the art, and those of ordinary skill in the art may analyze the liquid that washes the collecting tool according to the present invention without limitation.

### Example 1

1. A cotton swab was inserted as a collecting tool into a collecting device as shown in FIG. 3.

2. A pump connected to the rear section of the collecting device was operated to suck air at 10 L/min, thereby collecting microorganisms on the cotton swab.

3. The cotton swab was removed from the collecting device after the pump was powered-off.

4. The cotton swab was inserted into the bioluminescence kit.

5. A mixture of the cotton swab and a reagent in a kit was sufficiently shaken for a reaction.

6. The kit was inserted into a commercially available luminometer (ATP luminometer).

7. Relative light intensity was measured by the luminometer to determine a microbial concentration and calculate collection efficiency, and the collection efficiency was 99%.

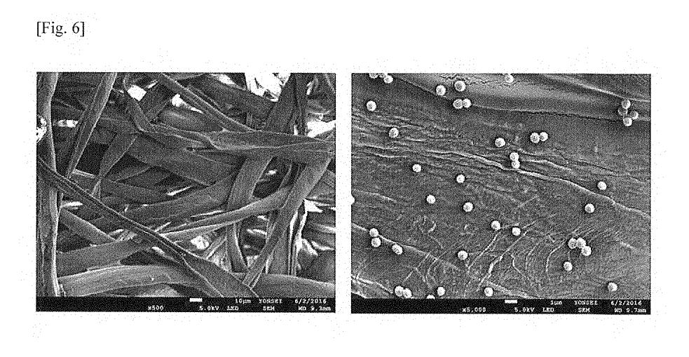

FIG. 6 shows an SEM image (left) taken before bacteria (Staphylococcus aureus) are collected on a cotton swab and an SEM image (right) taken after bacteria are collected on a cotton swab.

### Example 2

A process was performed in the same manner as described in Example 1, except air was sucked at 150 L/min, and a collection efficiency was 99%.

According to the present invention, all types of microorganisms in air can be easily and effectively collected and applied to various analysis devices. In addition, a conventional collecting device is expensive and requires highly skilled techniques, but a collecting device according to the present invention is small and has simple processing, and can be easily and simply used by common people without special expertise. Accordingly, since the device and method according to the present invention can easily collect microorganisms in air, which have recently attracted a great deal of attention, and can be analyzed by various analysis devices, there will be much demand therefor.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

### DESCRIPTION OF REFERENCE NUMERALS

10: first body
11: air inlet
12, 22: concave part
13, 23: air hole
14, 24: groove
15, 25: screw hole
20: second body
21: air outlet
30: sealing means
40: screw
50: collecting tool
51: collecting section
60, 61: connecting tube
70: fixing means
71: latch
72: lever
73: fixing bracket

What is claimed is:

1. A portable bioaerosol collecting device comprising:
a first body comprising an air inlet;
a second body coupled to the first body and comprising an air outlet; and
a collecting region formed by coupling the first and second bodies.

2. The device according to claim 1, wherein the first body or second body further comprises a fixing means for fixing the coupling of the first and second bodies.

3. The device according to claim 2, wherein the fixing means comprises a latch engaging with a latching jaw formed at a first or second body by latch-coupling, and a lever for forming and releasing the latch-coupling of the latch.

4. The device according to claim 2, wherein the fixing means comprises screw holes formed in the first and second bodies, and screws for screw-coupling with the screw holes.

5. The device according to claim 1, wherein the collecting region is created by coupling a concave part formed in the coupling surface of each of the first and second bodies.

6. The device according to claim 1, further comprising a collecting tool inserted into the collecting region.

7. The device according to claim 6, wherein the collecting tool is a cotton swab or a filter.

8. The device according to claim 1, wherein the first body includes an air hole configured to connect the air inlet and the collecting region, and the second body includes an air hole configured to connect the collecting region and the air outlet.

9. The device according to claim 1, wherein the first and second bodies include a groove formed in the coupling surface, and the device further includes a sealing means inserted into the groove.

10. The device according to claim 9, wherein the sealing means is a rubber ring.

11. The device according to claim 1, further comprising a pump connected to the air outlet or the air inlet.

12. The device according to claim 1, wherein a collection efficiency (E_{collection}), represented by Mathematical Formula 1 below, is in a range of 95 to 100%:
E_{collection}=[1−(N_{after}/N_{before})]×100 [Mathematical Formula 1]
wherein N_{before} refers to a microbial concentration (#/m³) in the front section of the collecting device, and N_{after} refers to a microbial concentration (#/m³) in the rear section of the collecting device.

13. The device according to claim 12, wherein the collection efficiency is in the range of 95 to 100% even at a high flow rate of 100 to 200 L/min.

14. A method of analyzing bioaerosols, comprising:
collecting bioaerosols by inputting air onto a bioaerosol collecting tool which is placed in a collecting region in the portable bioaerosol collecting device of claim 1; and
analyzing the bioaerosols collected on the bioaerosol collecting tool.

15. The method according to claim 14, wherein the inputting of air is performed using a pump.

16. The method according to claim 14, wherein, in the analysis step, only bioaerosols are extracted from the collecting tool using a lysis reagent, and then light intensity is measured, thereby quantifying microorganisms.

17. The method according to claim 16, wherein light intensity is measured using an adenosine triphosphate (ATP) bioluminescence kit.

18. The method according to claim 14, wherein, in the analysis method, the collecting tool is washed with a liquid, and then the liquid is subjected to mass spectrometry.

19. The method according to claim 18, wherein the mass spectrometry is performed using a Matrix-Assisted Laser Desorption and Ionization-Time of Flight (MALDI-TOF) mass spectrometer.

20. The method according to claim 14, wherein, in the analysis step, the collecting tool is washed with a liquid, and then the liquid is subjected to polymerase chain reaction (PCR) analysis.
